(19) United States
(12) Patent Application Publication
(10) Pub. No.: US 2021/0243185 A1
(43) Pub. Date: Aug. 5, 2021

(54) Tap and Go PHI System

(71) Applicant: Yogesh Narayan Thasale, San Diego, CA (US)

(72) Inventor: Yogesh Narayan Thasale, San Diego, CA (US)

(21) Appl. No.: 16/946,338

(22) Filed: Jun. 17, 2020

Related U.S. Application Data
(60) Provisional application No. 62/968,002, filed on Jan. 30, 2020.

(51) Int. Cl. H04L 29/06; G06K 7/14; G16H 10/60; G06F 21/62; G06F 21/60

(52) U.S. Cl. CPC H04L 63/0861; G06K 7/1413; G06F 21/602; G06F 21/6245; G16H 10/60

(57) ABSTRACT

An app which requests, and decrypts and stores, health record information from an encrypted QR code.

This application claims priority from Provisional application No. 62/968,002, filed Jan. 30, 2020, the entire contents of which are herewith incorporated by reference.

### BACKGROUND

Management of healthcare records can be a difficult endeavor for patients especially when changing doctors. Each new healthcare provider does not automatically get records from a previous provider. Due to confidentiality rules, such as HIPPA, prior healthcare records are stored in the prior provider's database, and a user must find some way to get those records from the prior provider to their new provider.

### SUMMARY OF THE INVENTION

The present application describes a system, including an application which can be run on a user's portable device such as a cellular phone or tablet, that allows a user to obtain their medical information from a provider, in a secure way.

In one embodiment, codes and/or the medical information can be provided at the end of their visit or time of discharge. The user can possess those codes, as part of their medical history.

In an embodiment, the medical information is provided by providing a barcode, here a first QR code. In embodiments, a QR code is used as the barcode, however, it should be understood that any computer scannable or readable code can be used in alternate embodiments.

The first QR code is used as a mechanism for identifying the patient. The patient's identity is verified as being associated with a profile that is related to the first QR code. Based on this verification, the information is used to obtain a second QR code that includes the data regarding the patient's health.

The QR codes (first and/or second) can be encrypted QR codes that is secure and can only be decoded by an encryption key. The encryption key can be stored in the user's device and operated by the app that is running in the user's device.

The medical information can be stored easily on the user's portable phone or tablet. In this way, the user receives access to all their health data in a secure way.

According to an embodiment, a high density barcode, here a QR code, is used to encode the data in a special way that can only be decoded responsive to receiving the user's own personal credentials. Once decoded, the information can be stored on the user's phone, or in a repository, e.g, a cloud account associated with the user's phone.

### BRIEF DESCRIPTION OF THE DRAWINGS

In the Drawings:

FIG. 1 shows a users receiving a scan of an encrypted barcode from a provider terminal;

FIG. 2 shows the scan appearing on the user's phone;

FIG. 3 shows a flowchart of operation;

FIG. 4 shows a functional diagram; and

FIG. 5 shows a communication diagram.

### DETAILED DESCRIPTION

The present application describes a system, referred to herein referred to as the tap and go PHI System, that allows a user to get their health records from their provider. In an embodiment, the records are obtained, for example, at the end of each visit. By obtaining the records in this way, the user has a record of their own healthcare information.

In embodiments, the health data obtained can include one or more of diagnosis, pharmacy information, treatment plan, prescriptions, lab reports, x-rays, as well as any other medical information for the current visit, or for any period of time.

In an embodiment, at the time of discharge, the user usually visits the medical receptionist to check out and/or pay their bill. According to an embodiment, the user is given an opportunity to scan and obtain their information. In one embodiment, the user runs an application on their phone, which is programmed according to the techniques described herein.

At the end of the visit, an electronic display, which can be a tablet, provider terminal or other display, at the provider's premises, shown as 100, has a display 101 which displays a readable code 105, in an embodiment an encrypted QR code, the first QR code.

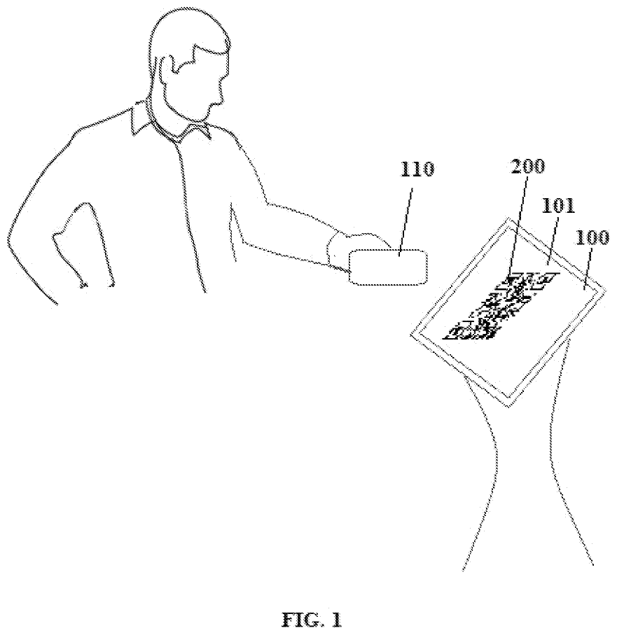

FIG. 1 shows the user uses their own personal device 110 to read the first QR code 105, using the camera on their personal device.

The first QR code 105 represents the healthcare customer, doctor's name, and information about the specific provider terminal

Upon capturing the image, the app obtains information from the user's profile, and also biometric information about the user of the app, obtained from the user. The app passes the information from the barcode (“image data”), along with the profile and user information, to a remote database associated with a third party provider, other than the medical information (“Company”), along with the customer's personal data. The Company receives the image data and looks up to determine which specific provider terminal initiated the request. If the personal data matches to the user data, the Company initiates a communication channel with the specific provider terminal. Once the channel is established, the provider terminal receives some personal data from the Company database and also some personal data info from the mobile app. This is used for verification.

Once all partys' data has been reviewed and verified (Healthcare Consumer, Provider Terminal, and Doctor), a communication channel is established with the Doctor's healthcare system to instruct release of the actual healthcare data. This healthcare data is received by the Provider Terminal. The provider terminal then creates a second QR Code 200 using that received health data. The user's phone is used to scan that second QR code 200.

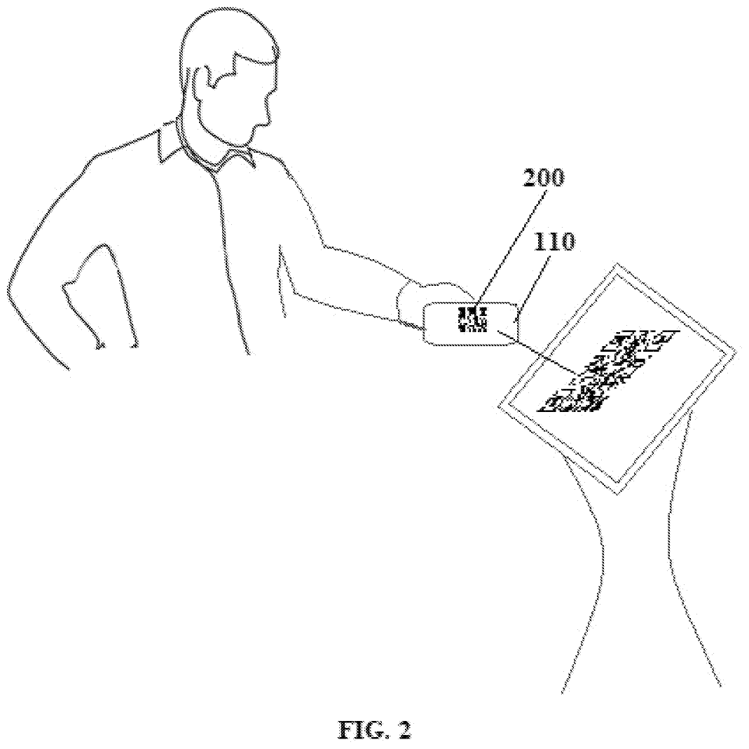

After reading, the second QR code 200 shows on the user's device 110 as shown in FIG. 2.

For medical privacy reasons, the health information in the QR code is encrypted. At this point, the app running on the mobile device carries out a security verification authentication, to make sure that the user of the app is the authorized user. This can use the identification verification structure that is already part of a user's phone, e.g., the face id in an apple or android phone. Verifying the identification of the user has the effect of enabling decoding of the data from the second QR code, thus allowing the user to see and store their own health data in the app, as received from the QR code. Only the authorized party can decrypt the data at the target.

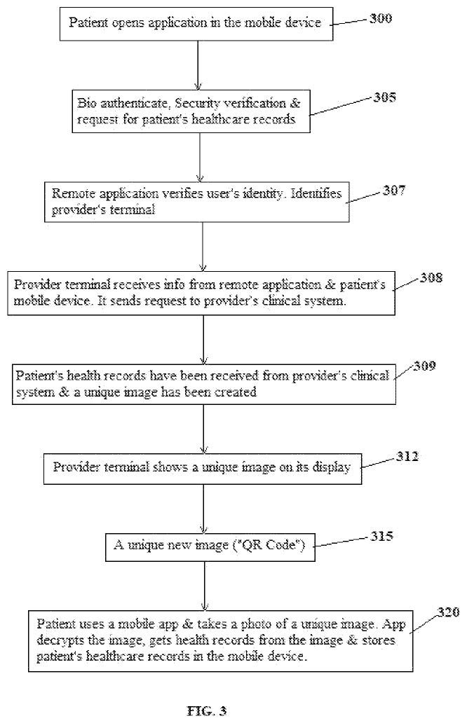

All of this is done according to the flowchart of FIG. 3, as explained herein.

At 300, the user opens the app on their phone, and carries out biometric authentication and security verification. This biometric authentication, for example, can leverage the existing hardware and software authentication systems in the phone, for example using face ID or the like with an iPhone. In alternate embodiments, this may also use additional authentication, such as security questions, or require entry of a PIN, or other multiple different forms of authentication if even further security is required.

At 306, User uses the app to scan the first QR code which is displayed in one embodiment, the QR code is displayed at the provider terminal 105. This is taken as a request to transfer health records from the app.

Upon receiving the user's request of the transfer of records, the Remote Server verifies the user at 307, and identifies the location/source of the provider terminal. This sends a job request for transfer of medical records to the provider terminal.

In an embodiment, communication between the User and Provider terminal, and between the (Remote Server 500 to provider terminal) are secure and uses two different communication paths.

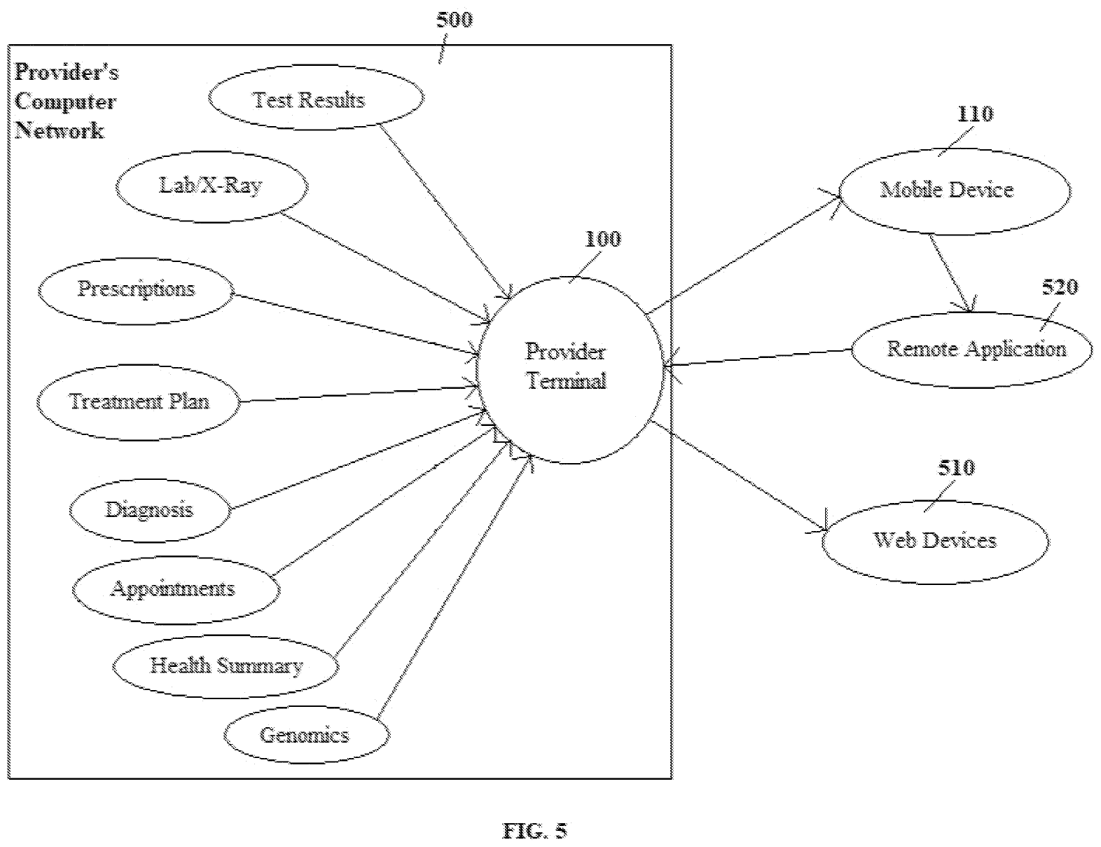

In a preferred embodiment, the remote server 500 is not owned by and not managed by Medical provider and it is not part of Medical Provider's infrastructure but is able to communicate to the provider terminal 100 over the secure communication channel. The remote server can be generally shown as 500 in FIG. 5 which shows the data communication of the provider terminal 100.

Upon receiving the request of health records transfer from the remote server, the provider terminal requests the User's health records from Medical Provider's Clinical systems at 308.

At 309, the provider terminal receives the User's health records from the Medical Provider's Clinical systems and generates a new 2d QR code 315. The provider terminal displays a secure encrypted 2d QR code at 315. This new QR code 315 is then scanned then by the user's phone. The 2d QR code 315 uses a public/private key system, where the 2d QR code 315 is encrypted using the public code, and can only be decrypted using the private code which is possessed by the owner of the information, and stored on the app.

At 320, the app uses the private key to decrypt the 2d QR code, and stores the data from the 2d QR code, for example in the memory of the phone, or in a cloud account.

In an alternative embodiment, the information can be scanned from a QR code printed, for example, on a paper receipt. This can be used, for example, when the Consumer can not scan the QR code or there is no display in provider's location to show the first QR code. For example, when the user receives the paper receipt indicative of payment or indicative of the end of the visit, the receipt includes a QR code.

This follows the following work flow.

The Medical provider prints the QR Code on the patient's visit notes.

Upon receiving the document. Consumer opens the app and scan the QR code.

Upon scanning the QR Code, app will perform tasks to verify their identity using 2 factor authentication.

Upon consumer verification, the consumer receives the key to decrypt QR code.

Upon receiving the key, app decrypts QR code and health data has been recorded in Consumer APP.

In both embodiments, the QR codes are encrypted so that only the authorized user who has the authorized private key can decrypt the information in the QR code. Therefore, by scanning that QR code, the app user's mobile device receives the information upon verification.

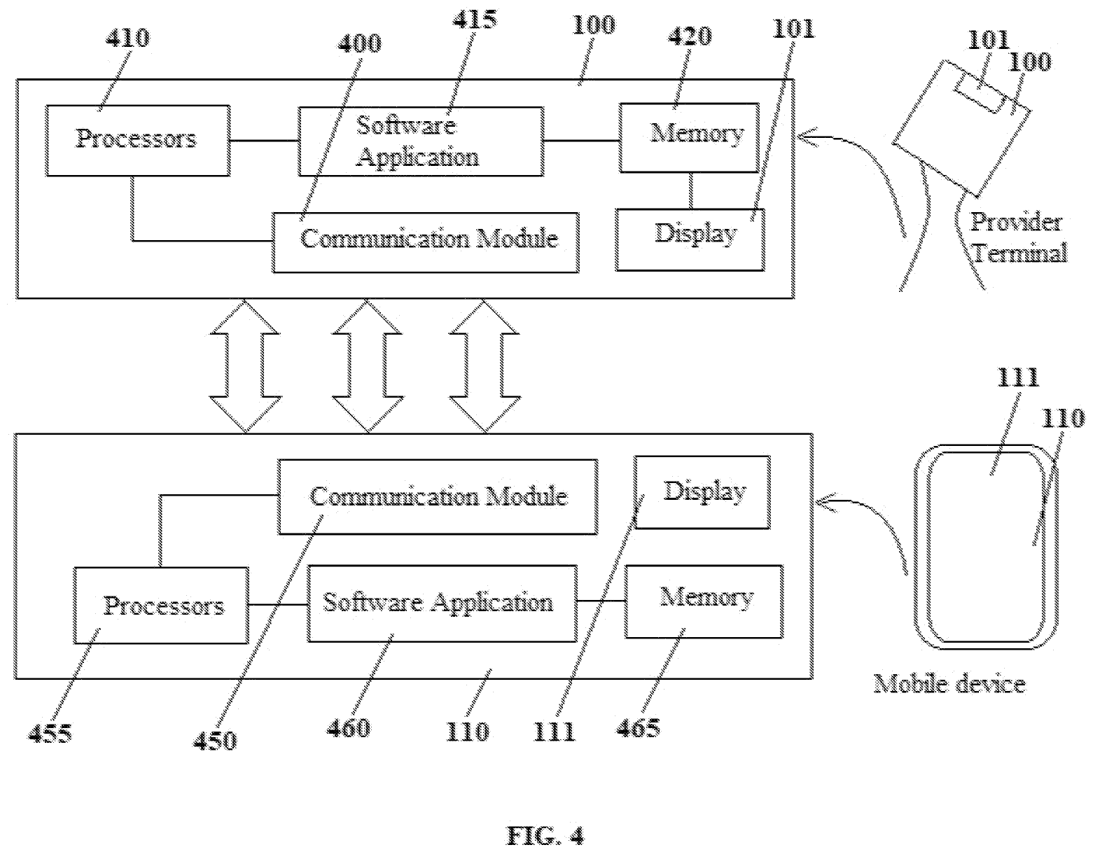

The hardware of the provider terminal and mobile device can be as shown in FIG. 4, The provider terminal has a communication module 400 which handles communication with a number of different items including the mobile device 110. The communication may be via Wi-Fi, Bluetooth or any wired or wireless technique. The communication module communicates with and is controlled by a processor 405 programmed to include applications 415, which operate based on information stored in memory 420. The information can be displayed on the display 101.

The mobile device 110 may also include a display 111 and may include a communication module 450, communicating with a processor 455, mobile application for 60 and memory 465.

FIG. 5 shows the different communication capability of the provider terminal, where the provider terminal 100 communicates with a number of different devices including the mobile device 110, but also with medical records storage structure shown generically as 500 which can include test results, labs x-rays, prescriptions, treatment plans, diagnoses, appointments, and health summaries. The provider terminal also can communicate with other web devices 510, as shown.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

1. A method of obtaining health care records, comprising:
at a location of a medical facility, using the portable device to request healthcare data; using a portable device to scan an image that is provided by the medical facility;
where the image is the visual representation of patient's healthcare data and is being created by software application that is running on provider computer network and the software application is implemented in combination with hardware, firmware, and as a set of instructions in software, residing on a memory and hard disk, and executed on a computer and other processing device. Each time it creates a new unique image for every patient's doctor visit and patient encounter,
using a mobile software application that is running on a patient's mobile device to obtain a patient's healthcare data from the image, and store the patient's healthcare data in the patient's portable device;
Where mobile software application is implemented in combination with hardware, firmware, and as a set of instructions in software, residing on a memory and hard disk, and executed on a mobile device.

2. The method as in claim 1, wherein requesting healthcare data comprises of Receiving patient's personal information from patient's mobile device to Remote application; Determining a specific provider terminal where patient had requested healthcare data; Opening a communication channel from Remote application to provider's computer network; Sending information about patient's personal information to the software application that is running on provider's computer network; Getting patient's healthcare data from the provider's clinical systems using patient's personal information by the software application that is running on the provider's network; Creating a unique image using patient's healthcare data by the software application that is running on the provider's network; Receiving information from the software application that is running on the provider's computer network.

3. The method as in claim 2, wherein information from the software application that is running on the provider's computer network comprises an image displayed on a provider terminal at the medical facility.

4. The method as in claim 1, wherein image is encoded and encrypted by the software application running on provider's computer network using patient's personal information

5. The method as in claim 4, wherein encoded and encrypted image is decrypted and decoded using a patient's personal information by the mobile software application that is running on a patient's mobile device.

6. The method as in claim 2, wherein a patient's personal information comprises personal identification information and security key information.

7. The method as in claim 1, wherein provider computer network comprises of provider terminal, kiosk, clinical information system, practice management system, patient administration system, pharmacy management system, medical imaging systems, medical lab information systems, medical billing system, clinical care systems, and network to facilitate data communication and data transfer between them.

8. The method as in claim 1, wherein the information is scanned from an image printed on the paper.

9. The method as in claim 8, further comprising, the portable device using a mobile software application that is running on a patient's mobile device to obtain a patient's healthcare data from the image, and store the patient's healthcare data in the patient's mobile device.

10-11. (canceled)

12. A method of obtaining health records from a medical facility, comprising:
using the user's portable device for scanning a QR code at a medical facility; where the QR is the visual representation of patient's healthcare data and is being created by software application that is running on provider computer network and the software application is implemented in combination with hardware, firmware, and as a set of instructions in software, residing on a memory and hard disk, and executed on a computer and other processing device. Each time it creates a new QR code for every patient's doctor visit and patient encounter,
using a mobile software application that is running on a patient's mobile device to obtain a patient's healthcare data from the QR code, and store the patient's healthcare data in the patient's portable device.
Where mobile software application is implemented in combination with hardware, firmware, and as a set of instructions in software, residing on a memory and hard disk, and executed on a mobile device.

13. The method as in claim 12, wherein QR code is encoded and encrypted by the software application running on the provider's computer network using the patient's personal information.

14. The method as in claim 13, wherein encoded and encrypted QR code is decrypted and decoded using a patient's personal information by the mobile software application that is running on a patient's mobile device.

15. The method as in claim 14, wherein patient's personal information comprises personal identification information and security key information.

16. The method as in claim 12, where the portable device is a portable phone.
